(19) United States
(12) Patent Application Publication
(10) Pub. No.: US 2026/0348893 A1
(43) Pub. Date: Nov. 5, 2026

(54) Device and method for detecting flame-retardant performance of tunnel flame-retardant asphalt

(71) Applicant: CHONGQING ARCHITECTURAL DESIGN INSTITUTE CO., LTD, Chongqing (CN)

(72) Inventors: Yue Ma, Chongqing (CN); Lu He, Chongqing (CN); Huiming Qu, Chongqing (CN); Yongkui Zhang, Chongqing (CN); Lulu Xu, Chongqing (CN); Saijun Cao, Chongqing (CN)

(73) Assignee: CHONGQING ARCHITECTURAL DESIGN INSTITUTE CO., LTD, Chongqing (CN)

(21) Appl. No.: 19/533,390

(22) Filed: Feb. 9, 2026

Related U.S. Application Data
(63) Continuation of application No. PCT/CN2025/092455, filed on Apr. 30, 2025.

(51) Int. Cl. G01N 25/00; G01N 33/42

(52) U.S. Cl. CPC G01N 25/00; G01N 33/42

(57) ABSTRACT

A device for detecting flame-retardant performance of tunnel flame-retardant asphalt includes a support frame, two insertion plates, a scoring tool, and a molding box, as well as a tray, a flame gun, and heating wires disposed on the support frame. The molding box is provided with a detachable bottom plate. Each insertion plate is provided with insertion posts and detachably disposed atop the molding box. An internal cavity of the molding box and a space between the insertion posts form a molding space for the flame-retardant asphalt. The molding box is supported on the tray. The scoring tool can form score lines on a top surface of a molded flame-retardant asphalt block. The heating wires can be inserted into blind holes formed in the molded flame-retardant asphalt block after removal of the insertion posts. The flame gun is positioned above the molding box and corresponds to a top opening.

### CROSS REFERENCE TO THE RELATED APPLICATIONS

This application is the continuation application of International Application No. PCT/CN2025/092455, filed on Apr. 30, 2025, the entire contents of which are incorporated herein by reference.

### TECHNICAL FIELD

The present disclosure relates to the field of municipal engineering, and in particular to a device and method for detecting flame-retardant performance of tunnel flame-retardant asphalt.

### BACKGROUND

Tunnels are engineering structures embedded in strata. Asphalt layers are paved on all road surfaces of the tunnels to reduce tire noise and dust emissions.

With a gradual increase in the ownership of new energy vehicles (NEVs), the probability of explosive combustion in batteries used by NEVs within tunnels is rising. After explosive combustion, the batteries can directly ignite the asphalt layers paved in the tunnels, causing the asphalt layers to burn and release large amounts of toxic gases. This poses significant hazards in enclosed environments of the tunnels. To mitigate such risks, flame-retardant asphalt is typically used inside the tunnels. After being ignited, the surface layer of the flame-retardant asphalt decomposes and releases gases to form a flame-retardant layer, thereby exhibiting flame-retardant properties. However, the flame-retardant asphalt layers paved in the tunnels are subjected to factors such as environmental temperature fluctuations and initial damage during long-term use, for example, high temperatures (tunnel temperatures in the Chinese city of Chongqing can reach 50 °C) and scratches. Therefore, it is necessary to detect changes in the flame-retardant performance of the paved flame-retardant asphalt. Additionally, municipal construction personnel need to know the minimum paving thickness required for the flame-retardant asphalt to achieve its flame-retardant effectiveness.

Therefore, designing a device with a simple structure and convenient detection for detecting the flame-retardant performance of flame-retardant asphalt is a technical problem that needs to be solved by those skilled in the art.

### SUMMARY

In view of the deficiencies of the prior art, a first objective of the present disclosure is to provide a device for detecting flame-retardant performance of tunnel flame-retardant asphalt. The device features a simple structure and low modification cost. It can simulate an extreme working condition of tunnel flame-retardant asphalt and test flame-retardant performance of the flame-retardant asphalt under the extreme working condition.

A second objective of the present disclosure is to provide a method for detecting flame-retardant performance of tunnel flame-retardant asphalt under an extreme working condition using the aforementioned device. The method features simple and convenient operation, and can accurately and objectively feed the flame-retardant performance of the flame-retardant asphalt back in accordance with practical requirements.

A technical solution to achieve the first objective of the present disclosure is as follows. A device for detecting flame-retardant performance of tunnel flame-retardant asphalt includes a support frame, insertion plates, a scoring tool, and a molding box, as well as a tray, a flame gun, and heating wires disposed on the support frame, where the molding box includes a lower box and an upper box connected to form an integrated structure; the lower box has a rectangular cross-section and is provided with a detachable bottom plate; the upper box has a trapezoidal cross-section and is provided with a top opening; sidewalls of legs of the upper box each are uniformly provided with a plurality of through holes; there are two insertion plates; each of the insertion plates is provided with a plurality of insertion posts; a number of the insertion posts corresponds to a number of the through holes in the sidewall of the leg; the insertion posts pass through the corresponding through holes, enabling the insertion plates to be detachably disposed atop the molding box; an internal cavity of the molding box and a space between the insertion posts form a molding space for the flame-retardant asphalt; the molding box is supported on the tray; the scoring tool is configured to form score lines on a top surface of a molded flame-retardant asphalt block; the heating wires are configured to be inserted into blind holes formed in the molded flame-retardant asphalt block after removal of the insertion posts; and the flame gun is positioned above the molding box and corresponds to the top opening.

The support frame is provided with a camera device, and a camera of the camera device is positioned above the molding box and corresponds to the top opening.

The scoring tool includes a mounting shaft and at least two blades assembled on the mounting shaft through a threaded fit; and two sides of each of the blades are respectively provided with locking nuts through the threaded fit.

Each of the blades has an arc-shaped structure and is provided with a serrated edge.

Two opposing sides at a bottom of the lower box are provided with insertion slots, respectively; and the bottom plate is connected between the two insertion slots via an insertion fit and forms a detachable connection with the lower box.

A center of an upper surface of the tray is coated red.

A technical solution to achieve the second objective of the present disclosure is as follows. A method for detecting flame-retardant performance of tunnel flame-retardant asphalt using the aforementioned device includes following steps:
1) pouring, after the bottom plate and the insertion plates are assembled in position, molten flame-retardant asphalt into the molding box, and allowing the molten flame-retardant asphalt to be naturally solidified and formed into the flame-retardant asphalt block;
2) disassembling the insertion plates, forming the blind holes in the flame-retardant asphalt block, and inserting, via the through holes, the heating wires into the blind holes formed in the flame-retardant asphalt block;
3) rotating the molding box by 180°, striking the bottom plate and a side plate to separate the flame-retardant asphalt block from an inner wall of the molding box, and disassembling the bottom plate;
4) reorienting the molding box, adjusting a spacing between adjacent blades of the scoring tool, and cutting score lines on the top surface of the flame-retardant asphalt block, and supporting the molding box on the tray;
5) heating the heating wires until a specified temperature is reached, removing the heating wires, turning on the flame gun to burn the top surface of the flame-retardant asphalt block, raising a surface temperature at a burning position on the top surface of the flame-retardant asphalt block to 350-450 °C in 10 s, holding the surface temperature for 50-60 s, and recording a change in the flame-retardant asphalt block until burning is complete;
6a) determining that the asphalt with a thickness and a type is unqualified if the top surface of the flame-retardant asphalt block inside the molding box expands upward and the upper surface of the tray is observed during this process;
6b) determining that the asphalt with a thickness and a type is unqualified if a depression appears on the top surface of the flame-retardant asphalt block inside the molding box; and
6c) determining that the asphalt with a thickness and a type is qualified if the top surface of the flame-retardant asphalt block inside the molding box expands upward and the upper surface of the tray is not observed during this process.

Furthermore, in the step 1), the flame-retardant asphalt includes following components:
85-90 wt % of asphalt;
10 wt % of a flame retardant; and
0-5wt % of a synergist;

where the synergist is one or more of montmorillonite, antimony oxide, a derivative of antimony oxide, zinc borate, and an organotin compound; the flame retardant comprises 60-90 wt % of hydroxide and 10-40 wt % of a mixture including halloysite nanotube and ferrocene in a mass ratio of 1:2 to 2:1; and the hydroxide is one or more of aluminum hydroxide, magnesium hydroxide, and calcium hydroxide.

Preferably, in the step 2), a straight line where the blind hole is located is at an angle of 35-55°, preferably 45°, with a horizontal plane; a length of a vertical projection of each blind hole is ¼ to ¾ of a height of the molding box; and in the step 4), 1 to 3 score lines are cut, each with a width being ⅙ to ¼ of a width of the opening of the upper box and a depth not exceeding ¼ of a thickness of the flame-retardant asphalt block.

Preferably, in the step 5), the specified temperature is 45-65 °C.

The above technical solutions have the following beneficial effects.
1. The device for detecting flame-retardant performance of tunnel flame-retardant asphalt includes a support frame, insertion plates, a scoring tool, a molding box, as well as a tray, a flame gun, and heating wires disposed on the support frame. The molding box includes a lower box and an upper box connected to form an integrated structure. The lower box has a rectangular cross-section and is provided with a detachable bottom plate. The upper box has a trapezoidal cross-section and is provided with a top opening. That is, the bottom of the molding box can be opened or closed via the bottom plate. Sidewalls of legs of the upper box are uniformly provided with a plurality of through holes. There are two insertion plates, and each of the two insertion plates is provided with a plurality of insertion posts. A number of the insertion posts corresponds to that of the through holes in the sidewalls of the legs. The insertion posts pass through the corresponding through holes, enabling each of the insertion plates to be detachably mounted atop the molding box. An internal cavity of the molding box and a space between the insertion posts collectively form a molding space for the flame-retardant asphalt. After the filled flame-retardant asphalt is formed into a block, the insertion plates are disassembled. The insertion posts in the insertion plates are withdrawn from the formed flame-retardant asphalt block, forming the blind holes in the formed flame-retardant asphalt block for mounting the heating wires. Moreover, the designed structure ensures normal mounting and disassembly of the insertion plates. The molding box is supported on the tray. The scoring tool is configured to form score lines on a top surface of the molded flame-retardant asphalt block, simulating damage formed during the use of a tunnel flame-retardant asphalt layer. The heating wires are configured to be inserted into the blind holes formed after removal of the insertion posts from the molded flame-retardant asphalt block. The heating wires are uniformly heated to heat the flame-retardant asphalt block, thereby simulating a high-temperature environment. The flame gun is positioned above the molding box and corresponds to the top opening. After the heating wires heat the flame-retardant asphalt block until a specified temperature is reached, the flame gun is turned on to burn a top surface of the flame-retardant asphalt block. Thus, the flame-retardant performance of the flame-retardant asphalt in a real environment is simulated, effectively meeting the requirements for detecting the flame-retardant performance of the tunnel flame-retardant asphalt.
2. The support frame is provided with a camera device, and a camera of the camera device is positioned above the molding box and corresponds to the top opening. During testing, a phenomenon of melting-through and accumulation may occur. The camera device captures a central area of the upper surface of the tray to determine whether melting-through occurs, ensuring the accuracy of test results.
3. The scoring tool includes a mounting shaft and at least two blades assembled on the mounting shaft via a threaded fit. Two sides of each of the blades are respectively provided with locking nuts through the threaded fit, enabling adjustment of the spacing between adjacent blades according to actual needs. The blades each feature an arc-shaped structure with a serrated edge. The serrated edge cut score lines of specific thickness and depth on the surface of the flame-retardant asphalt block. Alternatively, two or more blades are provided to cut score lines of required width, effectively meeting the detection requirements for the flame-retardant performance of tunnel flame-retardant asphalt.
4. In the method of the present disclosure, the flame-retardant asphalt block of specific shape is first molded via the molding box. The shaped asphalt block has a smaller projected area at the upper part and a larger projected area at the lower part. When a flame burns the upper surface of the flame-retardant asphalt block, the flame-retardant asphalt block simulates an actual distress environment where localized heating on the tunnel flame-retardant asphalt layer transfers heat downward, ensuring accurate feedback on flame-retardant performance. Additionally, score lines are formed on the surface of the flame-retardant asphalt block, and the flame-retardant asphalt block is heated to a specified level to simulate an extreme working condition of the tunnel flame-retardant asphalt layer, guaranteeing precise feedback on flame-retardant performance. The bottom plate of the molding box is disassembled, and the molding box is supported on the tray, and the formed accommodating space prevents overflow of the flame-retardant asphalt block after burning, which could cause inaccurate performance measurements and environmental contamination. Moreover, the central area of the tray's upper surface can be observed to determine whether there is melting-through in the flame-retardant asphalt, thereby avoiding false detection.
5. In the method of the present disclosure, the flame-retardant asphalt releases a gas to form a flame-retardant layer upon burning, thereby exhibiting flame-retardant properties. The flame-retardant performance of each flame-retardant asphalt block sample is evaluated based on the residual morphology after burning. The thickness of the qualified flame-retardant asphalt block is adjustable to determine the appropriate paving thickness. The method satisfies the practical need for laboratory-based determination of flame-retardant performance and paving thickness of the flame-retardant asphalt, while ensuring the accuracy of experimental results.

The present disclosure will be further described below with reference to the drawings and specific implementations.

### BRIEF DESCRIPTION OF THE DRAWINGS

FIG. 1 is a structural schematic diagram of the present disclosure;

FIG. 2 is an axonometric view of a molding box in the present disclosure;

FIG. 3 is a schematic diagram showing cooperation between the molding box and insertion plates in the present disclosure;

FIG. 4 is a schematic structural diagram of a scoring tool in the present disclosure;

FIG. 5 is a partial axonometric view of the scoring tool in the present disclosure;

FIG. 6 is a schematic diagram of step 1) in Embodiment 2;

FIG. 7 is a schematic diagram of step 2) in Embodiment 2;

FIG. 8 is a schematic diagram of step 4) in Embodiment 2;

FIG. 9 is a schematic diagram of a result in Embodiment 2;

FIG. 10 is a schematic diagram of a result in Embodiment 3; and

FIG. 11 is a schematic diagram of a result in Embodiment 4.

Reference Numerals: 1. support frame; 2. insertion plate; 21. insertion post; 3. scoring tool; 31. mounting shaft; 32. blade; 4. molding box; 41. lower box; 42. upper box; 43. bottom plate; 44. through hole; 45. insertion slot; 5. tray; 6. flame gun; 7. heating wire; 8. camera device; and 81. camera.

### DETAILED DESCRIPTION OF THE EMBODIMENTS

Embodiment 1

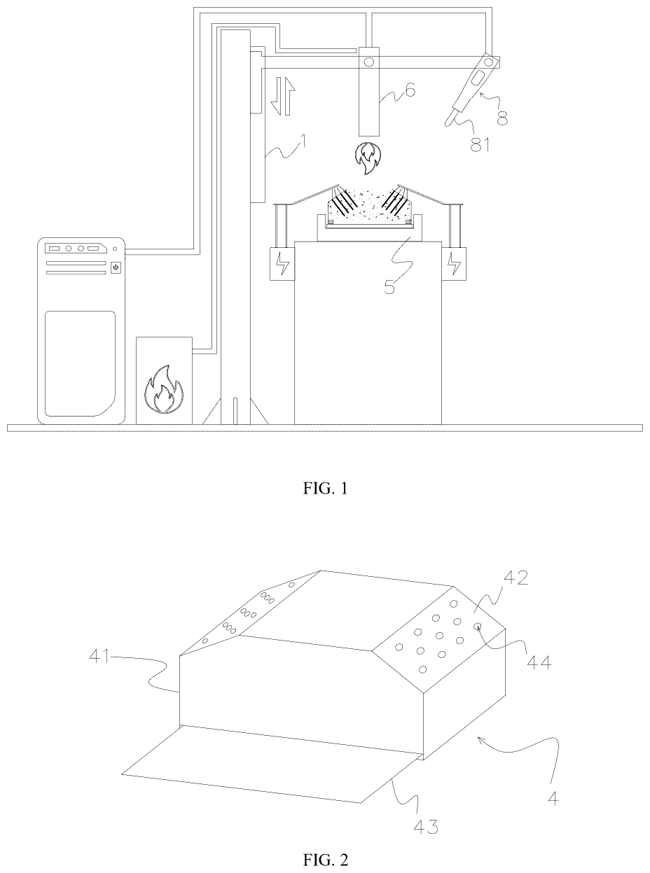

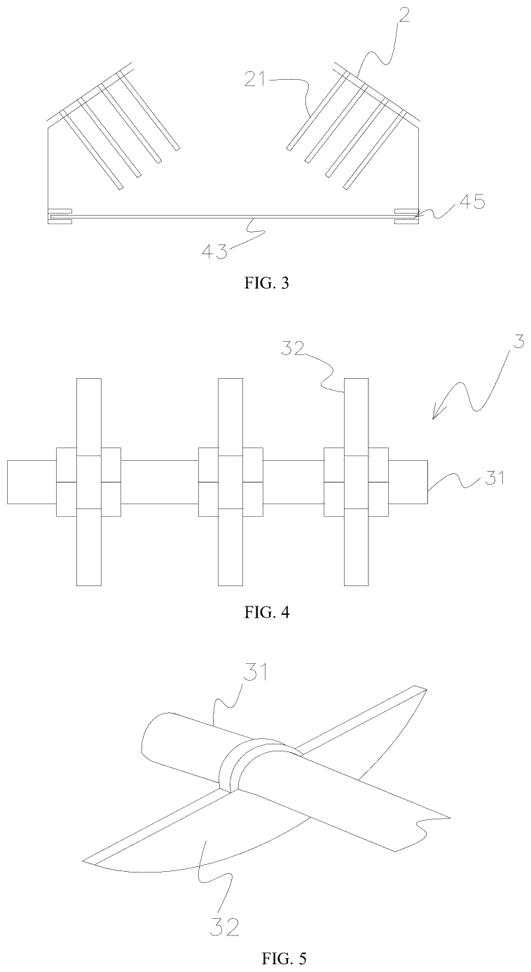

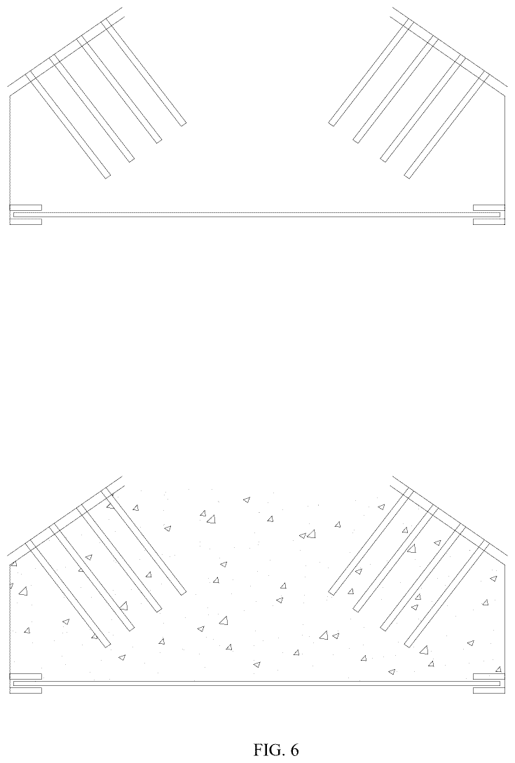

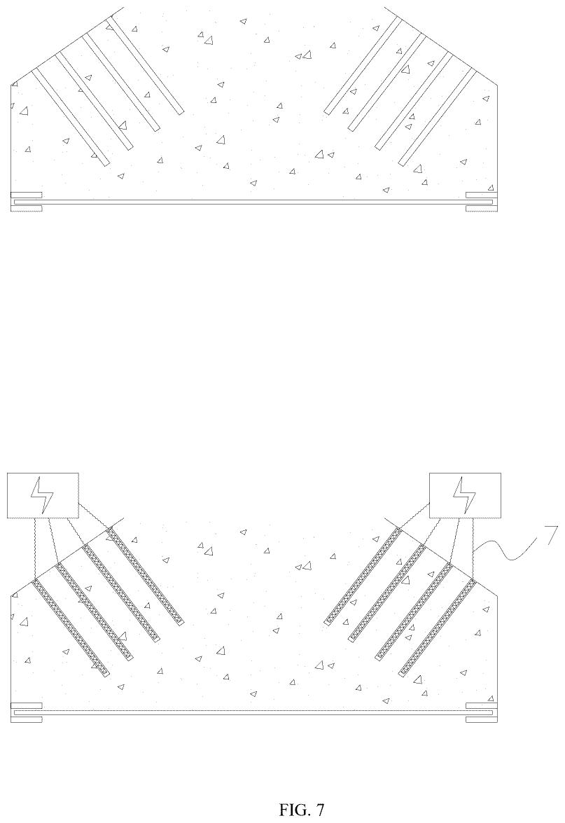

Referring to FIGS. 1 to 5, a device for detecting flame-retardant performance of tunnel flame-retardant asphalt includes support frame 1, insertion plates 2, scoring tool 3, and molding box 4, as well as tray 5, flame gun 6, and heating wires 7 disposed on the support frame. Specifically, the tray is made of an iron material. A center of an upper surface of the tray is coated red, forming a distinct contrast with a peripheral color. The scoring tool 3 includes mounting shaft 31 and three blades 32 assembled on the mounting shaft 31 through a threaded fit. Two sides of each blade 32 are respectively provided with locking nuts through the threaded fit. In this embodiment, each blade 32 has an arc-shaped structure and is provided with a serrated edge. The molding box 4 includes lower box 41 and upper box 42 connected to form an integrated structure. The lower box 41 has a rectangular cross-section and is provided with detachable bottom plate 43. Specifically, two opposing sides at a bottom of the lower box 41 are provided with insertion slots 45, respectively. The bottom plate 43 is connected between the two insertion slots 45 via an insertion fit and forms a detachable connection with the lower box 41. The upper box 42 has a trapezoidal cross-section and is provided with a top opening. Specifically, a sidewall of a leg of the upper box is at a 45° angle with a horizontal plane. The entire molding box has a height of 50 mm, a width of 50 mm, and a length of 100 mm. The sidewall of the leg of the upper box 42 is uniformly provided with through holes 44. Specifically, a single sidewall is provided with eleven through holes divided into three columns: a first column including four through holes, a second column including three through holes, and a third column including four through holes. There are two insertion plates 2. Each insertion plate 2 is provided with a plurality of insertion posts 21. A number of the insertion posts 21 corresponds to that of the through holes 44 in the sidewall of the leg. That is, each insertion plate is provided with eleven insertion posts, with the same distribution as the through holes. The insertion posts 21 pass through the corresponding through holes 44, enabling the insertion plate 2 to be detachably disposed atop the molding box 4. An internal cavity of the molding box and a space between the insertion posts collectively form a molding space for the flame-retardant asphalt. In this embodiment, a vertical projection of an extension length of each insertion post is ½ of the height of the molding box. That is, the vertical projection is 50 mm long. The molding box 4 is supported on the tray 5. The scoring tool 3 is configured to form score lines on a top surface of a molded flame-retardant asphalt block. The heating wires 7 are configured to be inserted into blind holes formed in the molded flame-retardant asphalt block after removal of the insertion posts. The flame gun 6 is positioned above the molding box 4 and corresponds to the top opening.

To ensure the accuracy of detection results, the support frame 1 is provided with camera device 8, and camera 81 of the camera device is positioned above the molding box 4 and corresponds to the top opening.

Embodiment 2

A method for detecting flame-retardant performance of tunnel flame-retardant asphalt uses the device of Embodiment 1. The flame-retardant asphalt includes following components:

85 wt % of asphalt;

10 wt % of a flame retardant; and

5 wt % of a synergist.

The synergist is montmorillonite. The flame retardant includes 90 wt % of hydroxide and 10 wt % of a mixture including halloysite nanotube and ferrocene in a mass ratio of 1:2. The hydroxide is calcium hydroxide.

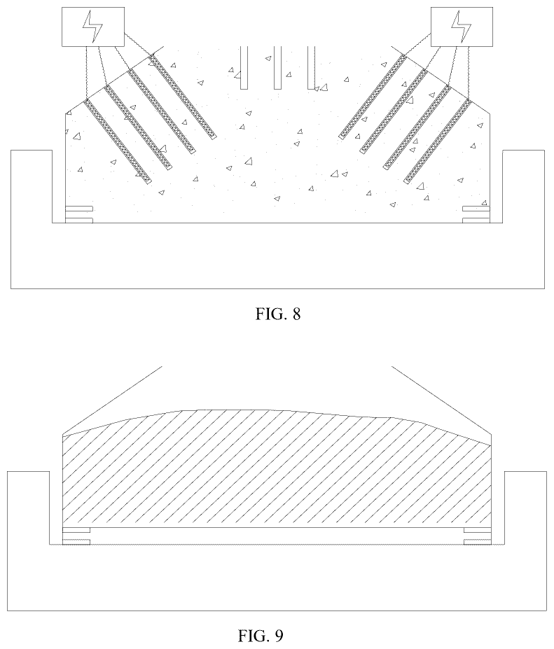

The method includes following steps.
1) After the bottom plate and the insertion plates are assembled in position, a molten flame-retardant asphalt is poured into the molding box. The molten flame-retardant asphalt is naturally solidified and formed into the flame-retardant asphalt block.
2) The insertion plates are disassembled, forming the blind holes in the flame-retardant asphalt block. The heating wires are respectively inserted into the blind holes formed in the flame-retardant asphalt block via the through holes.
3) The molding box is rotated 180°. The bottom plate and a side plate are struck to separate the flame-retardant asphalt block from an inner wall of the molding box. The bottom plate is disassembled.
4) The molding box is reoriented. A spacing between adjacent blades of the scoring tool is adjusted, and three score lines are made on a top surface of the flame-retardant asphalt block. The score lines extend across the flame-retardant asphalt block along a width direction. The molding box (including the flame-retardant asphalt block inside) is supported on the tray.
5) The heating wires are heated to 60 °C. The heating wires are removed, and the flame gun is turned on to burn the top surface of the flame-retardant asphalt block, raising a surface temperature at a burning position on the top surface of the flame-retardant asphalt block to 450 °C in 10 s. The surface temperature is held for 60 s. A change in the flame-retardant asphalt block is recorded until burning is complete.
6) If the top surface of the flame-retardant asphalt block inside the molding box expands upward and an upper surface of the tray is not observed during this process, the flame-retardant performance of the asphalt with a thickness and a type is determined qualified, as shown in FIGS. 6 to 9.

### Embodiment 3

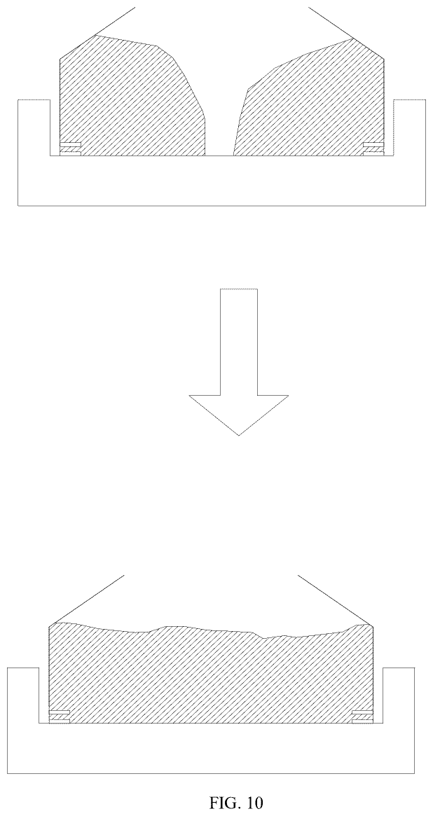

10 wt % of a commercial flame retardant, ammonium polyphosphate, is selected and added, and other operations are the same as Embodiment 2. The result is shown in FIG. 10. The top surface of the flame-retardant asphalt block inside the molding box expands upward, and the upper surface of the tray is observed during this process. This indicates that a melting-through phenomenon occurs during the burning process, and the flame-retardant performance of the asphalt with a thickness and a type is determined unqualified.

### Embodiment 4

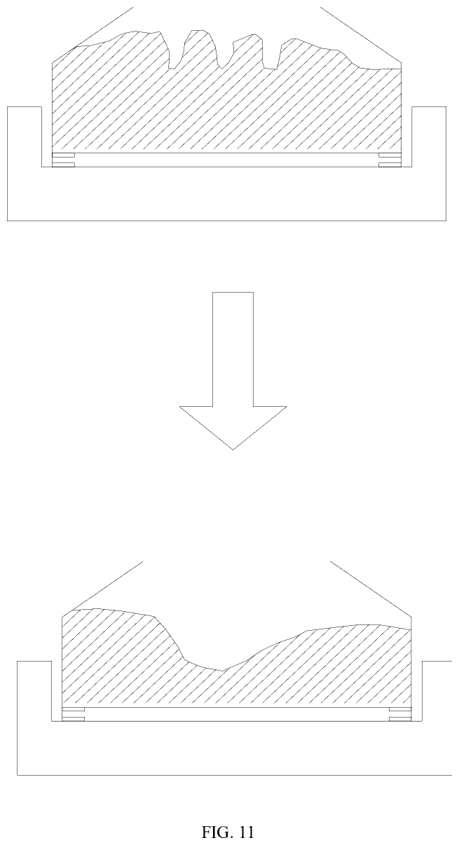

10 wt % of a commercial flame retardant, namely hydroxide, specifically a mixture of aluminum hydroxide and magnesium hydroxide in a weight ratio of 1:2, is selected and added, and other operations are the same as Embodiment 2. The result is shown in FIG. 11. A depression appears on the top surface of the flame-retardant asphalt block inside the molding box. This indicates that the flame-retardant layer formed by a gas released from the flame-retardant asphalt block upon burning has poor efficacy, and the flame-retardant performance of the flame-retardant asphalt at this thickness is determined unqualified.

1. A device for detecting a flame-retardant performance of a tunnel flame-retardant asphalt, comprising: a support frame, insertion plates, a scoring tool, and a molding box, as well as a tray, a flame gun, and heating wires disposed on the support frame, wherein
the molding box comprises a lower box and an upper box connected to form an integrated structure; the lower box has a rectangular cross-section and is provided with a detachable bottom plate; the upper box has a trapezoidal cross-section and is provided with a top opening;
and sidewalls of legs of the upper box each are uniformly provided with a plurality of through holes;
there are two insertion plates; each of the insertion plates is provided with a plurality of insertion posts; and a number of the insertion posts corresponds to a number of the through holes in the sidewall of the leg;
the insertion posts pass through the corresponding through holes, enabling the insertion plates to be detachably disposed atop the molding box; and an internal cavity of the molding box and a space between the insertion posts form a molding space for the flame-retardant asphalt;
the molding box is supported on the tray; the scoring tool is configured to form score lines on a top surface of a molded flame-retardant asphalt block; and the heating wires are configured to be inserted into blind holes formed in the molded flame-retardant asphalt block after removal of the insertion posts; and
the flame gun is positioned above the molding box and corresponds to the top opening.

2. The device for detecting the flame-retardant performance of the tunnel flame-retardant asphalt according to claim 1, wherein the support frame is provided with a camera device, and a camera of the camera device is positioned above the molding box and corresponds to the top opening.

3. The device for detecting the flame-retardant performance of the tunnel flame-retardant asphalt according to claim 1, wherein the scoring tool comprises a mounting shaft and at least two blades assembled on the mounting shaft through a threaded fit; and two sides of each of the blades are respectively provided with locking nuts through the threaded fit.

4. The device for detecting the flame-retardant performance of the tunnel flame-retardant asphalt according to claim 3, wherein each of the blades has an arc-shaped structure and is provided with a serrated edge.

5. The device for detecting the flame-retardant performance of the tunnel flame-retardant asphalt according to claim 1, wherein two opposing sides at a bottom of the lower box are provided with insertion slots, respectively; and the bottom plate is connected between the two insertion slots via an insertion fit and forms a detachable connection with the lower box.

6. The device for detecting the flame-retardant performance of the tunnel flame-retardant asphalt according to claim 1, wherein a center of an upper surface of the tray is coated red.

7. A method for detecting a flame-retardant performance of a tunnel flame-retardant asphalt, using the device according to claim 1, comprising following steps:
1) pouring, after the bottom plate and the insertion plates are assembled in position, a molten flame-retardant asphalt into the molding box, and allowing the molten flame-retardant asphalt to be naturally solidified and formed into the flame-retardant asphalt block;
2) disassembling the insertion plates, forming the blind holes in the flame-retardant asphalt block, and inserting, via the through holes, the heating wires into the blind holes formed in the flame-retardant asphalt block;
3) rotating the molding box by 180°, striking the bottom plate and a side plate to separate the flame-retardant asphalt block from an inner wall of the molding box, and disassembling the bottom plate;
4) reorienting the molding box, adjusting a spacing between adjacent blades of the scoring tool, and cutting score lines on the top surface of the flame-retardant asphalt block, and supporting the molding box on the tray;
5) heating the heating wires until a specified temperature is reached, removing the heating wires, turning on the flame gun to burn the top surface of the flame-retardant asphalt block, raising a surface temperature at a burning position on the top surface of the flame-retardant asphalt block to 350°C-450°C in 10 s, holding the surface temperature for 50-60 s, and recording a change in the flame-retardant asphalt block until burning is complete;
6a) determining that an asphalt with a thickness and a type is unqualified if the top surface of the flame-retardant asphalt block inside the molding box expands upward and an upper surface of the tray is observed during this process;
6b) determining that an asphalt with a thickness and a type is unqualified if a depression appears on the top surface of the flame-retardant asphalt block inside the molding box; and
6c) determining that an asphalt with a thickness and a type is qualified if the top surface of the flame-retardant asphalt block inside the molding box expands upward and the upper surface of the tray is not observed during this process.

8. The method according to claim 7, wherein in the step 1), the flame-retardant asphalt comprises following components:
85 wt %-90 wt % of asphalt;
10 w t% of a flame retardant; and
0-5 wt % of a synergist;
wherein the synergist is one or more of montmorillonite, antimony oxide, a derivative of antimony oxide, zinc borate, and an organotin compound; the flame retardant comprises 60-90 wt % of hydroxide and 10-40 wt % of a mixture comprising halloysite nanotube and ferrocene in a mass ratio of 1:2 to 2:1; and the hydroxide is one or more of aluminum hydroxide, magnesium hydroxide, and calcium hydroxide.

9. The method according to claim 7, wherein in the step 2), a straight line where the blind hole is located is at an angle of 35°-55°, preferably 45°, with a horizontal plane; a length of a vertical projection of each blind hole is ¼ to ¾ of a height of the molding box; and in the step 4), 1 to 3 score lines are cut, each with a width being ⅙ to ¼ of a width of the opening of the upper box and a depth not exceeding ¼ of a thickness of the flame-retardant asphalt block.

10. The method according to claim 7, wherein in the step 5), the specified temperature is 45°C-65°C.

11. The device for detecting the flame-retardant performance of the tunnel flame-retardant asphalt according to claim 2, wherein two opposing sides at a bottom of the lower box are provided with insertion slots, respectively; and the bottom plate is connected between the two insertion slots via an insertion fit and forms a detachable connection with the lower box.

12. The method according to claim 7, wherein in the device for detecting the flame-retardant performance of the tunnel flame-retardant asphalt, the support frame is provided with a camera device, and a camera of the camera device is positioned above the molding box and corresponds to the top opening.

13. The method according to claim 7, wherein in the device for detecting the flame-retardant performance of the tunnel flame-retardant asphalt, the scoring tool comprises a mounting shaft and at least two blades assembled on the mounting shaft through a threaded fit; and two sides of each of the blades are respectively provided with locking nuts through the threaded fit.

14. The method according to claim 13, wherein in the device for detecting the flame-retardant performance of the tunnel flame-retardant asphalt, each of the blades has an arc-shaped structure and is provided with a serrated edge.

15. The method according to claim 7, wherein in the device for detecting the flame-retardant performance of the tunnel flame-retardant asphalt, two opposing sides at a bottom of the lower box are provided with insertion slots, respectively; and the bottom plate is connected between the two insertion slots via an insertion fit and forms a detachable connection with the lower box.

16. The method according to claim 7, wherein in the device for detecting the flame-retardant performance of the tunnel flame-retardant asphalt, a center of an upper surface of the tray is coated red.
